(19) United States
(12) Patent Application Publication
(10) Pub. No.: US 2006/0253096 A1
(43) Pub. Date: Nov. 9, 2006

(54) Intelligent medical cabinet

(76) Inventors: Daniel Robert Blakley, Philomath, OR (US); Charles Bruce, Rochester, MN (US); Steven John Simske, Fort Collins, CO (US); Tong Zhang, San Jose, CA (US); Paul Friedman, Rochester, MN (US); Virend Somers, Rochester, MN (US)

Correspondence Address: HEWLETT PACKARD COMPANY, P O BOX 272400, 3404 E. HARMONY ROAD, INTELLECTUAL PROPERTY ADMINISTRATION, FORT COLLINS, CO, 80527-2400, US

(21) Appl. No.: 11/107,273

(22) Filed: Apr. 15, 2005

(51) Int. Cl. A61M 31/00

(52) U.S. Cl. 604/503; 604/65; 604/66

(57) ABSTRACT

A method for dynamic control of medications provided to a patient at a remote location is disclosed. The method can include steps of selecting a customized set of medications for a patient, where the customized set of medications at least partially reflect a medical diagnosis of the patient, and providing the customized set of medications to the patient, where the customized set of medications are inaccessible to the patient without authorization from a medical care provider. Further steps of the method can include authorizing the patient to receive a first medication regimen from the customized set of medications, monitoring remotely at least one health parameter of the patient, and authorizing the first medication regimen to be modified to a second medication regimen based at least partially on the at least one health parameter.

### FIELD OF THE INVENTION

The present invention relates generally to the monitoring and control of a patient's medication regimen at a remote location. More particularly, the present invention relates to the remote customization of a patient's medication regimen.

### BACKGROUND OF THE INVENTION

Hospitals provide certain types of medical care to many patients that are difficult to perform in a home environment. This is often the case with specific illnesses, surgeries, and other medical procedures that require a high level of monitoring of the patent, particularly the monitoring of medication consumption. It can often be necessary for patients to be monitored in a medical environment to insure that medication regimens are modified in response to certain physiological changes. When medical conditions improve to the point that only occasional medication monitoring is required, patients can be released from the hospital or medical facility. For many patients, however, improvement can be a slow process that requires hospitalization for extended periods of time. These prolonged stays can become detrimental to the health of many patients. Unhealthy bacteria and viruses are often concentrated in a hospital environment, increasing many patients' risks of contracting associated illnesses and diseases in relation to the length of their stay. Also, prolonged stays in such an environment begin to affect the mental well-being of many patients. They often may find it difficult to sleep, eat, and relax in such an environment. Similarly, monetary costs increase in relation to the length of the stay for patients, hospitals, and insurance providers.

As such, it is a benefit to all parties involved to discharge patients as soon as possible. In many cases, however, patients who would benefit from returning home are still in a condition that requires significant monitoring of medications. Discharging patients in this condition may create health risks to the patients and liability for hospitals and insurance providers. One solution is to monitor medication regimens on an outpatient basis. In these cases, patients can return to a hospital or other medical facility once or twice a week to allow a medical care provider to assess their physiologies and make modifications to their medication regimens. This may allow patients to be released to a more comfortable home environment while medication monitoring by hospitals continue. Through this procedure, hospitals can monitor the conditions of patients and take appropriate action when problems arise.

Several disadvantages arise with outpatient monitoring methods, however. One problem is related to the lower level of control that medical care providers have over patients in an outpatient setting. Many patients may be lax about returning to a medical facility for medication monitoring. Other patients, particularly the elderly, may have a difficult time with transportation. This is particularly a problem with monitoring that must occur frequently due to the relative seriousness of some medical conditions. Another problem is related to the response time of changes in a medication regimen to actual changes in patient's health. Many physiological changes can require a somewhat rapid modification of a medication regimen. In these cases, the speed of the modification is dependent on the medical care provider becoming aware of the physiological change, which will not occur until the patient returns to the outpatient facility. Because of these disadvantages, medical care providers may keep patients longer in a hospital environment, or they may send them home with an increased risk of further injury or even death. Early discharge can also lead to increased liability.

It would thus be helpful to devise a means of providing patients with sufficient medication monitoring such that early release from the hospital environment is possible, while at the same time allowing somewhat rapid changes in medication regimens due to patient monitoring.

### SUMMARY OF THE INVENTION

It has been recognized that a method of providing customized medication monitoring to a patient would be advantageous. It would also be beneficial for the medication monitoring to be customizable to specific circumstances surrounding a patient's medical treatment. Specifically, a method for dynamic control of medications provided to a patient at a remote location is disclosed. The method can include steps of selecting a customized set of medications for a patient, where the customized set of medications at least partially reflect a medical diagnosis of the patient, and providing the customized set of medications to the patient, where the customized set of medications are inaccessible to the patient without authorization from a medical care provider. Further steps of the method can include authorizing the patient to receive a first medication regimen from the customized set of medications, monitoring remotely at least one health parameter of the patient, and authorizing the first medication regimen to be modified to a second medication regimen based at least partially on the at least one health parameter.

In another embodiment of the present invention, a method for dynamic control of medications provided to a patient at a remote location is provided. The method can include steps of selecting a customized set of medications for a patient, and providing the customized set of medications to the patient, where the medications are inaccessible to the patient without authorization from a medical care provider. Further steps of the method can include selecting a first medication regimen from the customized set of medications, authorizing the patient to receive the first medication regimen, selecting a second medication regimen from the customized set of medications that is different from the first medication regimen, and authorizing the patient to receive the second medication regimen.

Additional features and advantages of the invention will be apparent from the following detailed description which illustrates, by way of example, features of the invention.

### DETAILED DESCRIPTION OF THE PREFERRED EMBODIMENTS

Before particular embodiments of the present invention are disclosed and described, it is to be understood that this invention is not limited to the particular process and materials disclosed herein as such may vary to some degree. It is also to be understood that the terminology used herein is used for the purpose of describing particular embodiments only and is not intended to be limiting, as the scope of the present invention will be defined only by the appended claims and equivalents thereof.

In describing and claiming the present invention, the following terminology will be used.

The singular forms “a,” “an,” and “the” include plural referents unless the context clearly dictates otherwise. Thus, for example, reference to “a medication” or a “medication regimen” includes reference to one or more of such modules.

As used herein, the term “medical care provider” refers to any individual providing medical care to a patient. This would include, for example, physicians, nurses, medical technicians, physicians assistants, pharmacists, lab technicians, etc.

As used herein, the term “secondary medical condition” refers to medical conditions arising in a patient that are unrelated, peripherally related, or can arise from treatment of the primary medical condition for which a patient is being monitored.

As used herein, the term “health parameter” refers to any aspect of a patient's physiology that can be monitored by a medical device. For example, a health parameter related to heart rhythms can include, without limitation, all electrocardiogram (ECG) waves, normal ECG waves, abnormal ECG waves, a portion of an ECG wave such as the QRS wave, etc.

The term “medication regimen” refers to a particular schedule of medication, including type, dosage, amount, timing, etc., being taken by a patient. The term “medication regimen” used without the antecedent “first” or “second” refers to a general medication regimen being used by the patient. As such, the terms “first medication regimen” and “second medication regimen” are used primarily to highlight a change in a medication regimen. Otherwise, all of these terms can be used interchangeably.

As used herein, the term “physiological waveform” refers to an analog or digital signal representation of a particular physiological activity. A physiological waveform can refer to a waveform of any given length. For example, physiological waveform can be an ECG recorded over several hours, a sequential series of ECGs from a particular episode, a set of non-sequential normal or abnormal ECGs, a single ECG, or a portion of an ECG such as a QRS wave.

The term “about” when referring to a numerical value or range is intended to encompass the values resulting from experimental error that can occur when taking measurements.

With these definitions in mind, it has been recognized that a method for dynamic control of medications provided to a patient at a remote location would be an advancement in the art. It would also be beneficial for the dynamic control of medications to be related to health monitoring of the patient.

Embodiments of the present invention provide methods for medication monitoring that may allow the early release of many patients from a hospital setting that would normally require in-hospital monitoring. These methods provide for take-home medication in an “intelligent medical cabinet” (IMC) that can respond to a patient's changing at-home medical condition, particularly where such changing medical conditions may warrant a change in a medication regimen. The IMC can provide the patient and the medical care provider with the benefit of insuring that a medication regimen remains appropriate for the extended periods of time that can lapse between the patient's medical care appointments. If a change in medication is required, the medical care provider can authorize a new medication regimen for the IMC to dispense that day. Such direct access of a patient's medication through the IMC allows a medical care provider a level of control previously obtained primarily only in a hospital environment. This direct access also may save the patient from return trips to the pharmacy, as well as providing a means of faster drug therapy changes.

Such beneficial medication control can be facilitated by the direct monitoring of particular health parameters associated with a patient's medical condition. Changes in these health parameters can be indicative of a changing medical condition, which can be corrected by modifications to the medication regimen. Such bidirectional communication between the patient and the medical care provider may facilitate early discharge, and thus may afford the medical care provider flexibility in managing patient care when the patient has left the direct care and supervision of a hospital, clinic, outpatient setting, or other medical facility.

The IMC can utilize a type of “recipe book” for conditional care that maps an initial prescription into the most likely future prescriptions of pharmaceuticals according to a patient's conditions and needs. In one aspect, it can be based on historical data for the same patient or patients with similar symptoms, diagnosis and/or prognosis. The idea behind such adaptive medicine is to provide customization of a patient's at-home medical care by sending home the most likely future pharmaceuticals that may be needed for that particular patient, given the patient's medical condition and past patient medical history. Because the pharmaceuticals located within the IMC are inaccessible to the patient without authorization, the medical care provider can control the medication regimen taken by the patient, and thus, more closely replicate the medication monitoring conditions of a hospital-like environment.

In one embodiment of the present invention, a method for dynamic control of medications provided to a patient at a remote location is disclosed. The method can include steps of selecting a customized set of medications for a patient, where the customized set of medications at least partially reflect a medical diagnosis of the patient, and providing the customized set of medications to the patient, where the customized set of medications are inaccessible to the patient without authorization from a medical care provider. The method can further include steps of authorizing the patient to receive a first medication regimen from the customized set of medications, monitoring remotely at least one health parameter of the patient, and authorizing the first medication regimen to be modified to a second medication regimen based at least partially on the at least one health parameter.

The step of selecting a customized set of medications for a patient can be performed by any means known to one skilled in the art. The customized set of medications can at least partially reflect a medical diagnosis of the patient. In one aspect, the selection of a customized set of medications can originate from a prescription by a qualified medical care provider. In such cases, the medical care provider can assess the patient and determine a diagnosis and/or other related aspects of a medical condition that may benefit from medications. From this determination, a prescription can be generated from which a set of medications can be selected that can be helpful in treating the medical condition. In one aspect, the medical care provider can check off medications from a checklist of various medications associated with a particular medical condition. In another aspect, the medical care provider can accept a suggested “stock prescription” selection associated with the medical condition. In yet another aspect, the medical care provider can generate a generalized prescription containing a description of the medical condition, and the set of medications can be selected automatically based on that description. The selection of a set of medications can also include those medications that may not be immediately relevant to the medical condition of the patient, but that the medical care provider determines may be of some use in the future treatment of the medical condition. As described above, the selection can also be based on historical data for the same patient or patients with similar symptoms, diagnosis and/or prognosis.

In addition to those medications that may be directly related to the medical condition, medications can be included in the selected set of medications that might be useful in treating potential secondary medical conditions that may arise in the future. For example, a patient following surgery may be sent home with an IMC loaded with a set of medications related primarily to the after effects of the surgery, such as narcotics, anti-inflammatories, and sleeping pills. Because it is possible that a secondary infection may arise in such cases, the medical care provider may include various antibiotic medications in the set of medications contained in the IMC. If a secondary infection is observed via the monitoring of the health of the patient, the antibiotics can be authorized by the medical care provider and they will be dispensed by the IMC.

Medications can also be included in the set of medications that are selected by the medical care provider based on previous medical histories of the patient. These medications can be related to past incidents known to have occurred to the patient. In these cases, the medications are included in order to be available in the event that these incidents arise again. Alternatively, or additionally, they can be medications that are routinely taken by the patient, thus allowing the IMC to simplify the patient's medication routine by dispensing all needed medications from a convenient source.

In one embodiment, the step of selecting a set of medications also may include selecting the dosage form of a particular medication. For example, a medical care provider can select a transdermal patch for a patient known to have trouble swallowing pills. Alternatively, the medical care provider can select pills for a patient known to have a skin condition that will not tolerate transdermal patches. In another embodiment, the step of selecting a set of medications also may include selecting the dosage amount for a particular medication. It is foreseeable that a change in dosage of a particular medication may be important in the future. If the IMC is loaded only with 500 mg pills of this medication, options may be limited for altering the dosage. If, on the other hand, various sizes of pills have been included, e.g., 500 mg, 250 mg, and 100 mg, the total dosage dispensed to the patient can be altered significantly. This can be helpful when increasing the medication dosage to combat a worsening medical condition. It can also be helpful when decreasing the medication dosage as the medical condition improves or as deleterious side effects such as tremors or palpitations occur.

It should be understood that the selection of the set of medications may not entirely be made by the medical care provider assessing the patient's medical condition. In some cases, the medical care provider may even be unaware of at least a portion selection procedure, having only written up a diagnosis or a prescription providing only general medical details of the condition. Other medical care providers, on the other hand, may be more intimately involved in the selection of the set of medications, including those related to secondary medical conditions and prior patient medical histories. In other words, the level of involvement of the medical care provider in the steps of the various methods disclosed herein may be highly variable, and may often depend on their medical expertise in a particular area, i.e. a generalist vs. a specialist.

The customized set of medications can be provided to the patient such that they are inaccessible to the patient without authorization from a medical care provider. In this way, a wide variety of medications can be provided to the patient without the worry that they will be taken inappropriately. In one aspect, the customized set of medications can be provided to the patient in a remotely operated medication lockbox that can be selectively unlocked to provide the patient access to various medication regimens. It is not intended, however, that the details concerning the dispensing of medications be limiting. Any means known to one skilled in the art of selectively dispensing medication to a patient at a remote location while maintaining at least a portion of the set of medications in an inaccessible state would be considered to be within the scope of the present invention.

As one example, the IMC is a medication strongbox or lockbox that delivers medications to a patient at preset time intervals. The time intervals can be constant, or they can vary, depending on the particular medication regimen in effect. The type and amount of each medication can vary from one time interval to the next. The IMC can deliver any type of medication known to one skilled in the art, including oral medications, parenterals, patches, creams, etc. As has been discussed above, the IMC requires authorization from the medical care provider before dispensing medication. Once authorization is received, a medication regimen can continue until the authorization expires or is revoked. For example, authorization may be given for single dose of a medication, a limited number of doses, a limited time period, etc. If the authorization is for two pills three times a day for three days, the IMC will dispense 18 pills over three days with the proper time intervals and then cease dispensing that medication. In one aspect, the type and amount of medication can vary from one time interval to the next. Dispensing can also be directly controlled from the medical care provider site for various safety reasons such as tampering, as is discussed below.

The IMC can be pre-loaded with the set of medications related to the patient's medical condition, potential secondary conditions, and prior patient medical histories. The IMC can also contain nonprescription medication such as pain relievers and antihistamines. The patient is prohibited from unauthorized medication consumption because the IMC will only dispense the appropriately prescribed type, dosage, and amount of each medication in a medication regimen each day. In one aspect, the patient is further protected in that modifications in the current medication regimen can only be authorized by a medical care provider though a secured verification means. Additionally, the IMC can be security protected, sending a signal to the medical care provider if it has been tampered with. The signal can include information concerning the extent of the tampering, including any dosage or prescription changes that have been made.

Medication consumption can be somewhat monitored by including a medication detector in a catch tray of the IMC. This detector can send a signal to the medical care provider when dispensed medication is removed from the catch tray. Additionally, aspects can also include a “retrieval and retention” mechanism in the catch tray to prevent dispensed medications from accumulating when they are not appropriately removed in a timely fashion. A sound can also be emitted from the IMC to notify the patient that new medication has been dispensed into the tray and is ready for use.

Once the patient has received the set of medications, the medical care provider can authorize the patient to receive a first medication regimen from the customized set of medications. The order of the authorization is not critical in this case, and can also occur prior to providing the patient the set of medications. Once the authorization is provided, the patient is allowed access to a limited dose of the medications of the first medication regimen.

In another embodiment of the present invention, at least one health parameter of the patient can be monitored. Such monitoring can provide some level of measurement as to the effectiveness of the first medication regimen, and also provide some indication as to potential medication alterations that would be beneficial in a second medication regimen. When a patient in need of monitoring is sent to a location remote from a hospital or other medical facility, various health parameters can potentially be monitored. In order to appropriately monitor a medication regimen, it can be helpful to monitor health parameters that would be linked to the medical condition of the patient in such a way as to provide useful indicators of the medication's effectiveness. A health parameter is thus merely a manifestation of a physiological process that is in some way indicative of some aspect of the health of the patient. The health parameter can be an objective measurement such as an electrocardiogram (ECG) or a blood pressure reading, or it can be a subjective determination such as an indication from the patient that they feel light-headed. The health parameter can also include a mental assessment of the patient in order to monitor and dispense mood altering or other neurological medications.

An intended health parameter can be determined at various levels. In one aspect, the intended health parameter can be determined through a prescription communicated by the medical care provider. Various means of assisting in this determination are available, regardless of the involvement of the medical care provider. In one aspect, a checklist for a particular medical condition can be utilized to suggest potential intended health parameters, with particular emphasis on those health parameters effected by a given medication regimen. This checklist can allow a medical care provider to check off specific intended health parameters to monitor while the patient is on a particular medication regimen. For example, and without limitation, a checklist for heart related medical conditions can include monitoring suggestions for specific hardware needs, sensors to be used for the type of diagnosis monitored, such as atrial fibrillation, congestive heart failure, premature ventricular contraction, etc. Also, the checklist may or may not be technical in nature. For example, one type of checklist may provide suggested hardware, software, data analysis, etc. Another type may provide more mundane suggestions, such as the ambulatory level of the patient, the quality of the stored data, the amount of data to save, specific portions of an ECG wave to examine etc. Monitoring of health parameters could then be selected based on these more mundane suggestions. It is also contemplated that a suggested checklist could be provided to the medical care provider for their acceptance. These checklists can also be crafted and stored for future use with patients with similar medical conditions.

The selection of intended health parameters may also be based on a prior medical history of the patient or of patients with similar symptoms, diagnosis, prognosis, etc. It may be useful to include a monitored health parameter related to secondary medical conditions that have arisen in the past, thus allowing the medical care provider the notice to dispense medications related to secondary medical conditions. Also, the selection can include health parameters related to medical conditions that the medical care professional believes may arise, or desires to monitor in order to have enough advance warning of subsequent medical issues to authorize a second medication regimen.

The monitored health parameter can be selected at any scale recognizable to one skilled in the art. For example, the intended health parameter can be heart rate, the timing of contractions of the heart, or a particular sequence of the contraction cycle. Through the selection of these particular intended health parameters, a medical monitoring device can be constructed to monitor heart rate, ECG waveforms, or specific sections of ECG waveforms, respectively. Thus it is contemplated that monitored health parameters also include portions of physiological waveforms, such as the QRS portion of the ECG waveform. Monitored health parameters can also include specific types of data selected from an overall group. For example, the medical care provider can specify that the health parameter is “all abnormal ECGs”, and thus normal ECGs can be discarded or compressed to conserve space in the medical monitoring device, while abnormal ECGs be left uncompressed and either stored on the device or sent directly to a medical monitoring facility.

The monitoring of at least one health parameter allows a medical care provider to quickly authorize a second medication regimen for the IMC to dispense to the patient, with the intention that the second medication regimen may improve the health of the patient, at least as to the monitored health parameter(s). The authorization of the first medication regimen to be modified to a second medication regimen can thus be based at least partially on the monitored health parameter(s). The modification of medication regimens can be the addition of at least one medication, the subtraction of at least one medication, or the complete change of all medications in the first medication regimen to new medications in the second medication regimen. The modification of medication regimens can also include changes in the dosage of one or more medications in the first medication regimen. Also, the modification can include changes in the timing of particular medications in the first medication regimen. For example, if it appears that a patient is metabolizing a particular medication too quickly and thus may have inadequate blood serum concentration of the pharmaceutical of interest, the dosage can be increased or the time interval between dispensings can be decreased. Thus the second medication regimen would improve the effectiveness of the medication therapy by increasing blood serum levels of the drug. Thus the authorizing of the first medication regimen to be modified to the second medication regimen allows the patient access to a limited dose of medications of the second medication regimen.

In another embodiment of the present invention, a second medication regimen can be selected that is different from the first medication regimen. Following the selection of the second medication regimen, the patient can be authorized to receive it. In one aspect the authorizing of the patient to receive the second medication regimen can occur when the first medication regimen is stopped. In another aspect, the authorizing can occur simultaneously with the first medication regimen.

In one embodiment of the present invention, the monitoring of the patient via at least one health parameter and any subsequent modification of the first medication regimen to a second medication regimen is carried out between a medical care provider in a hospital or other medical facility and a remote location such as the home of the patient. The authorization of the first medication regimen can occur similarly, or it can occur prior to the patient receiving the set of medications in the IMC. As such, in one aspect, remote monitoring and authorization for either medication regimen can be carried out electronically, particularly over a network. The network can be any type of communications network known to one skilled in the art, such as, without limitation, the Internet, local area networks, cellular networks, telecommunications networks, dedicated telecommunication lines, cable networks, digital satellite networks, Zigbee, Bluetooth, other wireless means, etc.

It is intended that two-way communication be established between the medical care provider and the patient being monitored. The two-way communication can utilize the same network in both directions, or it may utilize a different network in each direction. For example, two-way communication can be accomplished solely via an Internet connection, or it can be accomplished by a combination of Internet and cellular network connections. Communication networks are well known to those skilled in the art, and as such they will not be discussed in detail herein.

One advantage of two-way communication is that it allows the medical care provider the ability to monitor the health of a patient and immediately modify the medication regimen that the patient is receiving, with little or no input from the patient; the patient simply utilizes the medications that are dispensed by the IMC. Also, changes in medication often can increase certain health risks. The two-way communication allows the subsequent monitoring of the second medication regimen for potential medication side effects and to monitor their effectiveness.

As a hypothetical example of one embodiment of the present invention, a patient diagnosed with a heart-related medical condition can be released from a hospital with an ECG Holter type heart monitor for monitoring atrial fibrillation and with an IMC. The set of medications typically used for atrial fibrillation that may be pre-loaded into the IMC may include beta-blockers, diuretics, angiotensin-converting enzyme (ACE) inhibitors, angiotensin 11 receptor blockers (ARB), calcium channel blockers, alpha blockers, and central-acting agents such as central adrenergic inhibitors. The IMC could then be pre-loaded with various other medications depending on the patient's relative condition, including the possibilities of secondary medical conditions and medications related to the patient's past medical history. Patient medical history may also facilitate a modification of the heart-related medications pre-loaded into the IMC. For example, the patient may be at risk for beta-blockers. Beta-blockers can have negative side effects in combination with medicines used to treat high blood pressure, certain anti-depressants, allergy shots, diabetic medications, and medications used to treat asthma. In this case, the IMC should be pre-loaded with alternative medications that avoid these side effects. Once the remote monitoring of the atrial fibrillation has begun, the medical care provider can authorize the type and dosage of atrial fibrillation medication to be dispensed as a medication regimen based on the amount of atrial fibrillation; its progression to congestive heart failure, hypertension, or angina; or its disappearance.

It is to be understood that the above-referenced arrangements are illustrative of the application for the principles of the present invention. Numerous modifications and alternative arrangements can be devised without departing from the spirit and scope of the present invention while the present invention has been described above in connection with the exemplary embodiments(s) of the invention. It will be apparent to those of ordinary skill in the art that numerous modifications can be made without departing from the principles and concepts of the invention as set forth in the claims.

What is claimed is:

1. A method for dynamic control of medications provided to a patient at a remote location, comprising steps of:
selecting a customized set of medications for a patient, the customized set of medications at least partially reflecting a medical diagnosis of the patient;
providing the customized set of medications to the patient, the customized set of medications being inaccessible to the patient without authorization from a medical care provider;
authorizing the patient to receive a first medication regimen from the customized set of medications;
monitoring remotely at least one health parameter of the patient; and
authorizing the first medication regimen to be modified to a second medication regimen based at least partially on the at least one health parameter.

2. The method of claim 1, wherein at least one of the steps of authorizing is carried out electronically.

3. The method of claim 2, wherein at least one of the steps of authorizing is carried out over a network.

4. The method of claim 1, wherein the step of selecting the customized set of medications further includes steps of:
generating a medical prescription based at least partially on the medical diagnosis; and
selecting the customized set of medications based on the medical prescription.

5. The method of claim 1, wherein the step of selecting the customized set of medications further includes selecting a stock prescription based on the medical diagnosis.

6. The method of claim 1, wherein the step of selecting the customized set of medications further includes selecting at least a portion of the customized set of medications based on potential secondary medical conditions.

7. The method of claim 1, wherein the step of selecting the customized set of medications further includes selecting at least a portion of the customized set of medications based on a prognosis of the patient.

8. The method of claim 1, wherein the customized set of medications is provided to the patient in a remotely operated medication lockbox that can be selectively unlocked to provide the patient access to various medication regimens.

9. The method of claim 1, wherein the step of monitoring remotely is carried out over a communication network.

10. The method of claim 1, wherein the at least one health parameter includes a physiological measurement.

11. The method of claim 1, wherein the at least one health parameter includes a subjective description by the patient.

12. The method of claim 1, wherein the at least one health parameter includes a mental assessment of the patient.

13. The method of claim 1, wherein the step of authorizing the first medication regimen from the customized set of medications allows the patient access to a limited dose of medications of the first medication regimen.

14. The method of claim 1, wherein the step of authorizing the first medication regimen to be modified to the second medication regimen allows the patient access to a limited dose of medications of the second medication regimen.

15. A method for dynamic control of medications provided to a patient at a remote location, comprising steps of:
selecting a customized set of medications for a patient;
providing the customized set of medications to the patient, the medications being inaccessible to the patient without authorization from a medical care provider;
selecting a first medication regimen from the customized set of medications;
authorizing the patient to receive the first medication regimen;
selecting a second medication regimen from the customized set of medications that is different from the first medication regimen; and
authorizing the patient to receive the second medication regimen.

16. The method of claim 15, wherein the step of authorizing the patient to receive the second medication regimen occurs when the first medication regimen is stopped.

17. The method of claim 15, wherein the step of authorizing the patient to receive the second medication regimen occurs simultaneously with the first medication regimen.

18. The method of claim 15, further comprising a step of monitoring remotely at least one health parameter of the patient.

19. The method of claim 18, wherein the step of selecting the second medication regimen is based at least partially on the at least one health parameter.

20. The method of claim 15, wherein at least one of the steps of authorizing is carried out electronically.

21. The method of claim 20, wherein at least one of the steps of authorizing is carried out over a network.

22. The method of claim 15, wherein the step of authorizing the patient to receive the first medication regimen allows the patient access to a limited dose of medications of the first medication regimen.

23. The method of claim 15, wherein the step of authorizing the patient to receive the second medication regimen allows the patient access to a limited dose of medications of the second medication regimen.
